(19) United States
(12) Patent Application Publication
(10) Pub. No.: US 2026/0306693 A1
(43) Pub. Date: Oct. 8, 2026

(54) PORTABLE OZONE GENERATOR FOR BIOCIDAL AIR AND SURFACE TREATMENT

(71) Applicant: HS Ultra LLC, Cameron, WI (US)

(72) Inventors: Alex Haag, Cameron, WI (US); Brent Spurling, Luck, WI (US)

(21) Appl. No.: 19/636,204

(22) Filed: Apr. 1, 2026

Related U.S. Application Data
(60) Provisional application No. 63/782,389, filed on Apr. 2, 2025.

(51) Int. Cl. A61L 9/12

(52) U.S. Cl. CPC A61L 9/122; A61L 2209/14; A61L 2209/212

(57) ABSTRACT

Various embodiments of the present technology generally relate to a portable ozone generator that includes a housing defining an opening extending between an air intake side and an air discharge side. A fan is positioned within the opening to draw ambient air through the intake side and propel it toward the discharge side, while a filter upstream of the opening conditions the incoming air. An ozone generation module is arranged around the perimeter of the opening converts oxygen in the ambient air to ozone at production rates ranging from approximately 60 grams per hour to 384 grams per hour. When operated, the generator increases ozone concentration within an enclosed area of up to 10,000 cubic feet to levels between about 4 parts per million and 70 parts per million within approximately 15 to 45 minutes, providing a compact, high‑output solution for rapid ozone treatment.

### TECHNICAL BACKGROUND

This disclosure generally relates to portable ozone generation systems, and more specifically, to high-output ozone generators capable of achieving biocidal concentrations using ambient air while maintaining portability.

### BACKGROUND

Ozone is an oxidizing agent with well-documented efficacy for disinfection, odor mitigation, and microbial inactivation across residential, commercial, agricultural, and industrial settings. Conventional ozone generation systems fall into two broad categories: small, low-output consumer devices that plug into standard electrical outlets but produce ozone concentrations sufficient only for light deodorization and incapable of achieving bacterial lethality; and large, industrial-scale systems that are trailer-mounted or skid-mounted, weigh hundreds of pounds, require specialized electrical service or oxygen concentrators, and produce ozone at concentrations high enough to degrade materials, damage gaskets, and harm plastics and electronics. The industrial systems, while effective for disinfection, present numerous challenges for field deployment due to their size, weight, power requirements, and the need to distribute high-concentration ozone rapidly across large spaces before it reverts to oxygen.

### SUMMARY

Technology disclosed herein includes a portable ozone generator having a housing that defines an opening extending between an air intake side and an air discharge side. A fan is positioned within the opening and draws ambient air into the air intake side and propels the ambient air toward the air discharge side. An ozone generation module is positioned within the housing and arranged around a perimeter of the opening. The ozone generation module converts oxygen in the ambient air to ozone at a production rate within a range between 60 grams per hour and 384 grams per hour. A filter is positioned upstream of the ozone generation module on the air intake side. When operated, the portable ozone generator elevates an ozone concentration within an area to between 4 parts per million (PPM) and 70 PPM. In some embodiments, the area is up to 10,000 cubic feet, while in other embodiments, the area is greater than 10, 000 cubic feet. In some implementations, the ozone concentration within the area is elevated within 15 minutes to 45 minutes.

In an example embodiment, the ozone generation module includes multiple ozone generation units spaced equidistantly about the perimeter of the opening. The ozone generation units may be corona discharge ozone generation units.

In another example embodiment, the fan has a flow rate between 1800 and 4600 cubic feet per minute when operated. The portable ozone generator may draw power from standard electrical circuitry of between 120 volts and 220 volts at less than 15 amps.

In another example embodiment, the housing has dimensions configured for manual transport, including a width of between 15 inches to 35 inches, a height of between 15 inches to 35 inches, a depth of between 5 inches to 15 inches, and a weight of up to 50 pounds. In other embodiments, the housing has a weight of over 50 pounds and may include attached wheels to allow for transport. The housing may include a hinged door on the air intake side that securely attaches to a first side of the housing and permanently attaches to a second side of the housing.

In another example embodiment, the portable ozone generator includes a control system for maintaining the ozone concentration within the area at a preset concentration. The control system includes an ozone sensor, a run timer, and a toggle switch for selecting from multiple fan speed settings. The control system may further include a wireless communication module configured to enable communication via Bluetooth, WiFi, or a local area network. The wireless communication module enables coordination between multiple portable ozone generators deployed within a facility.

Technology disclosed herein also includes a method for treating an enclosed area with ozone. The method includes positioning a portable ozone generator within an area, where the portable ozone generator has a housing defining an opening extending between an air intake side and an air discharge side, a fan positioned within the opening, and an ozone generation module arranged around a perimeter of the opening. The method further includes drawing ambient air into the air intake side of the housing using the fan, filtering the ambient air upstream of the ozone generation module, and converting oxygen in the ambient air to ozone using the ozone generation module at a production rate within a range between 60 grams per hour and 384 grams per hour. The method also includes propelling ozone-containing air from the air discharge side into the area and elevating an ozone concentration within the area of up to 10,000 cubic feet to between 4 PPM and 70 PPM, in some cases within 15 minutes to 45 minutes.

Technology disclosed herein further includes a portable ozone generator where the ambient air limits a maximum ozone concentration producible by the portable ozone generator. The portable ozone generator achieves an ozone concentration sufficient for microbial inactivation within a treatment zone.

This Summary is provided to introduce a selection of concepts in a simplified form that are further described below in the Detailed Description. It may be understood that this Summary is not intended to identify key features or essential features of the claimed subject matter, nor is it intended to be used to limit the scope of the claimed subject matter.

### BRIEF DESCRIPTION OF THE DRAWINGS

Many aspects of the disclosure can be better understood with reference to the following drawings. The components in the drawings are not necessarily drawn to scale. Moreover, in the drawings, like reference numerals designate corresponding parts throughout the several views. While several embodiments are described in connection with these drawings, the disclosure is not limited to the embodiments disclosed herein. On the contrary, the intent is to cover all alternatives, modifications, and equivalents.

FIGS. 1A, 1B, and 1C provide perspectives of an example portable ozone generator, according to an embodiment herein.

FIG. 2 provides an illustration of a method for operating a portable ozone generator, according to an embodiment herein.

FIG. 3 provides a blow-out perspective of an example portable ozone generator, according to an embodiment herein.

FIGS. 4A and 4B provide perspective of another example portable ozone generator, according to an embodiment herein.

FIGS. 5A, 5B, 5C, 5D, and 5E provide varying perspectives of another example portable ozone generator, according to an embodiment herein.

### DETAILED DESCRIPTION

Ozone (O₃) is a highly reactive triatomic form of oxygen that functions as a potent oxidizing agent. When introduced into air or onto surfaces, ozone reacts with cellular membranes, viral envelopes, proteins, nucleic acids, and other organic structures, leading to the inactivation or destruction of bacteria, viruses, mold, fungi, spores, and other microbial contaminants. In this disclosure, a biocidal air and surface treatment refers to a deliberate process of elevating ozone concentration within an enclosed or partially enclosed area to levels sufficient to inactivate or kill microbial organisms present in the air, on exposed surfaces, or within porous materials. Because ozone reacts directly with biological targets without leaving residues, it has been used for decades in applications such as odor removal, indoor environmental sanitation, food‑processing sterilization, greenhouse and agricultural treatment, and industrial disinfection cycles. These properties make ozone a desirable agent for achieving rapid microbial inactivation across residential, commercial, agricultural, and industrial environments.

Existing technologies for generating ozone generally fall into two broad categories: (1) contractor‑grade portable ozone generators that operate on ambient air and plug into standard 120‑volt electrical outlets, and (2) industrial oxygen‑fed ozone systems that rely on oxygen concentrators or bottled oxygen to produce extremely high‑purity ozone. Contractor‑grade portable ozone generators are lightweight units often marketed for household deodorization, pet‑odor mitigation, and general odor control. These devices typically rely on low‑output ozone‑producing elements and limited air‑handling capability, which restrict the volume of ambient air they can process and the amount of ozone they can introduce into an enclosed space. As a result, such devices generate only modest ozone mass flow—sufficient for light deodorization but inadequate for achieving microbial inactivation or any meaningful room‑scale biocidal treatment.

Industrial oxygen‑fed systems, by contrast, rely on high‑purity oxygen streams and large‑capacity discharge modules. These systems are frequently mounted on trailers or skids, weigh hundreds of pounds, require dedicated electrical circuits or multi‑phase industrial power, and can produce ozone concentrations approaching one million parts per million (PPM), far exceeding the concentrations suitable for safe use within typical built environments.

Each of these existing categories presents numerous technical limitations that impede effective deployment for routine biocidal treatment. Contractor‑grade portable units produce relatively small ozone quantities and typically require extended treatment times—often six hours or more—to achieve even modest increases in ozone concentration within a single bedroom. These long exposure times, combined with the inability to achieve biocidal ozone concentrations, make such units unsuitable for time‑sensitive or high‑volume treatment scenarios common in commercial, agricultural, or industrial settings. Industrial oxygen‑fed systems, although capable of extremely high ozone output, present the opposite problem: they produce ozone at concentrations so high that they can degrade plastics, gaskets, sealants, sensitive electronics, and building materials. They also require oxygen concentrators, high‑power electrical infrastructure, and controlled distribution systems to prevent material damage. Furthermore, because the ozone they generate is extremely concentrated near the source, distributing that ozone uniformly across a large facility before it decays is technically challenging and often infeasible in practice.

Intermediate systems larger than household devices but smaller than industrial skid‑mounted systems have been attempted but inherit many of the same fundamental limitations. Increasing the number of corona‑discharge plates in a portable form factor commonly results in electrical saturation, uneven discharge behavior, overheating, and airflow collapse. These systems still rely on limited ambient‑air oxygen content and do not incorporate the air‑handling capacity required to sustain high throughput. As such, even when multiple mid‑scale units are deployed simultaneously, treatment cycles remain long, ozone levels remain well below biocidal thresholds, and coverage across large spaces such as barns, warehouses, or manufacturing floors is inconsistent. In many field applications, dozens or even hundreds of such units are needed to achieve performance approaching that of a single industrial oxygen‑fed system, rendering these approaches impractical

The technical challenge in improving portable, ambient‑air‑fed ozone generators lies in scaling ozone mass output while maintaining stable airflow and safe operating characteristics. When a fan cannot move ozone‑laden air away from the generation region quickly enough, a stagnant pocket of high ozone concentration forms around the generator, reducing the availability of oxygen that can be converted into additional ozone. This stagnation limits the achievable ozone concentration and accelerates degradation of corona‑discharge components through thermal stress and dielectric breakdown. High‑output industrial systems avoid this problem by using oxygen concentrators that supply high‑purity oxygen to the discharge modules, but such systems are too large, heavy, and power‑intensive to be considered portable. Attempts to increase the output of contractor‑grade units by adding additional discharge plates or stacking modules generally result in rising internal temperatures, non‑uniform electric field distribution, increased arcing risk, and reduced airflow due to upstream filter loading and geometric restrictions. Field testing has shown that the actual ozone mass delivery of such up‑scaled portable systems is often significantly lower than advertised, with performance deteriorating rapidly as thermal and electrical stresses compound during prolonged operation.

To address at least these shortcomings of conventional ozone generation systems, a portable ozone generator is provided herein. As will be described in greater detail below, the portable ozone generator provided herein is designed to rapidly generate and distribute ozone at concentrations suitable for biocidal air and surface treatment while using only ambient air as the oxygen source. The device draws ambient air into the housing, propels the air across an ozone generation module arranged around a perimeter of an internal opening, and converts oxygen in the incoming airflow into ozone using high‑efficiency corona‑discharge surfaces. Because the system relies solely on ambient air, the available oxygen inherently limits the maximum achievable ozone concentration, enabling ozone levels that are effective for microbial inactivation without reaching the extremely high, material‑damaging concentrations characteristic of oxygen‑fed industrial systems. The ozone‑containing air is then discharged into a treatment zone—an enclosed or partially enclosed environment in which ozone disperses and accumulates to levels sufficient to perform a biocidal treatment. The unit operates on standard residential or industrial electrical circuitry and does not require an oxygen concentrator, bottled oxygen, or specialized power infrastructure. The characteristics of such a treatment zone, and the conditions under which biocidal concentrations are established, are described in greater detail below.

Ozone generation technology described herein enables biocidal air and surface treatment within a defined treatment zone by elevating ozone concentrations rapidly and uniformly throughout the space. As used in this disclosure, “biocidal air and surface treatment” refers to the introduction and maintenance of ozone within an environment at concentrations and durations sufficient to inactivate microbial organisms present in the air, on exposed surfaces, and within porous or semi‑porous materials. Microbial inactivation is achieved through ozone’s oxidative action, which disrupts lipid membranes, protein structures, nucleic acids, and other biological components of bacteria, viruses, mold, fungi, spores, and similar contaminants. In many applications, ozone concentrations of approximately 4 to 70 PPM, maintained for a period of time dependent on a target of disinfection or cleanliness, are adequate to produce measurable microbial reduction or complete inactivation depending on environmental conditions and surface characteristics.

During a biocidal treatment, ozone disperses throughout the air volume of a room or structure and diffuses across surfaces it contacts. Because ozone is a reactive gas, the achievable level of microbial inactivation is tied to both concentration and exposure time. Many biological targets exhibit inactivation behavior that correlates to a concentration‑time (C×t) relationship, where higher concentrations for shorter periods can achieve the same effect as lower concentrations for longer periods. Conventional disinfection literature and field measurements indicate that ozone concentrations between approximately 4 parts PPM and 70 PPM, when maintained for periods depending on a target of disinfection or cleanliness, are generally sufficient to achieve meaningful microbial reduction or full inactivation depending on the material and environmental conditions. These concentrations are far higher than those produced by consumer deodorizing ozone devices yet far lower than the extreme, material‑degrading concentrations generated by industrial oxygen‑fed ozone systems.

Within this disclosure, an enclosure treated by the device is referred to as a treatment zone. A treatment zone may include an entire room, a barn or animal housing structure, an industrial processing area, or a defined portion of a larger enclosure. The treatment zone encompasses the volume into which ozone is introduced and within which the ozone is dispersed and held at a concentration sufficient for biocidal effect. In practice, the treatment zone may span up to approximately 10,000 cubic feet, up to 20,000 cubic feet, or up to 30,000 cubic feet for a single portable unit, although multiple units may be deployed cooperatively in larger facilities to establish overlapping ozone fields and uniform treatment coverage.

The portable ozone generator described herein is configured to generate and distribute ozone at production rates far exceeding those of small consumer ozone units while avoiding the weight, complexity, and infrastructure burdens associated with industrial oxygen‑fed systems. In operation, the device draws ambient air into a housing using a high‑volume fan and propels the air across an ozone generation module arranged around a perimeter of an opening within the housing. The ozone generation module converts oxygen in the ambient air to ozone through corona‑discharge surfaces, generating what is effectively a high‑throughput ozone stream without requiring bottled oxygen or an oxygen concentrator. Because the system uses only ambient air, the maximum achievable ozone concentration is inherently limited by the available oxygen fraction in the incoming air. This ambient‑air‑limited behavior permits the device to achieve concentrations in the biocidal range while avoiding extreme, unsafe levels characteristic of oxygen‑fed systems.

Once generated, ozone‑containing air is discharged into the treatment zone and distributed by the fan velocity and the unit’s volumetric flow rate. The combination of ozone production rate and air‑moving capacity enables rapid saturation of the treatment zone to the target ozone concentration. The portable ozone generator is designed for use in real‑world environments—including agricultural barns with airborne particulates, industrial spaces with open volumes, and residential or commercial rooms—while operating on standard 120‑volt or 220‑volt electrical service drawing less than 15 amps. The system thereby provides a portable, high‑output, ambient‑air‑fed ozone generator capable of achieving biocidal concentrations quickly, safely, and without requiring specialized electrical or oxygen‑feed infrastructure.

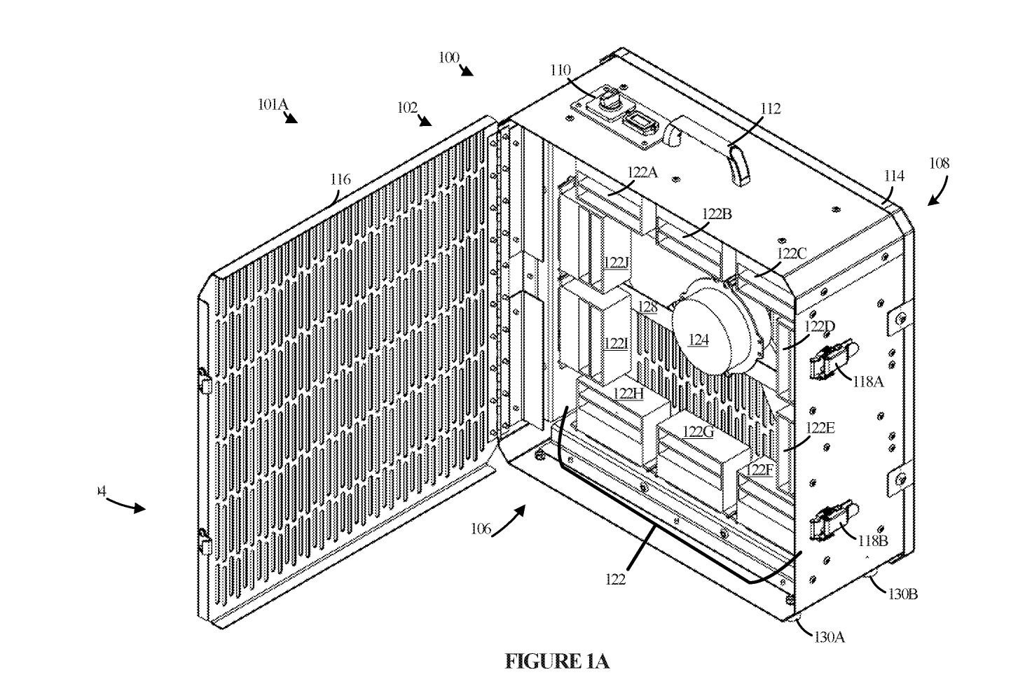

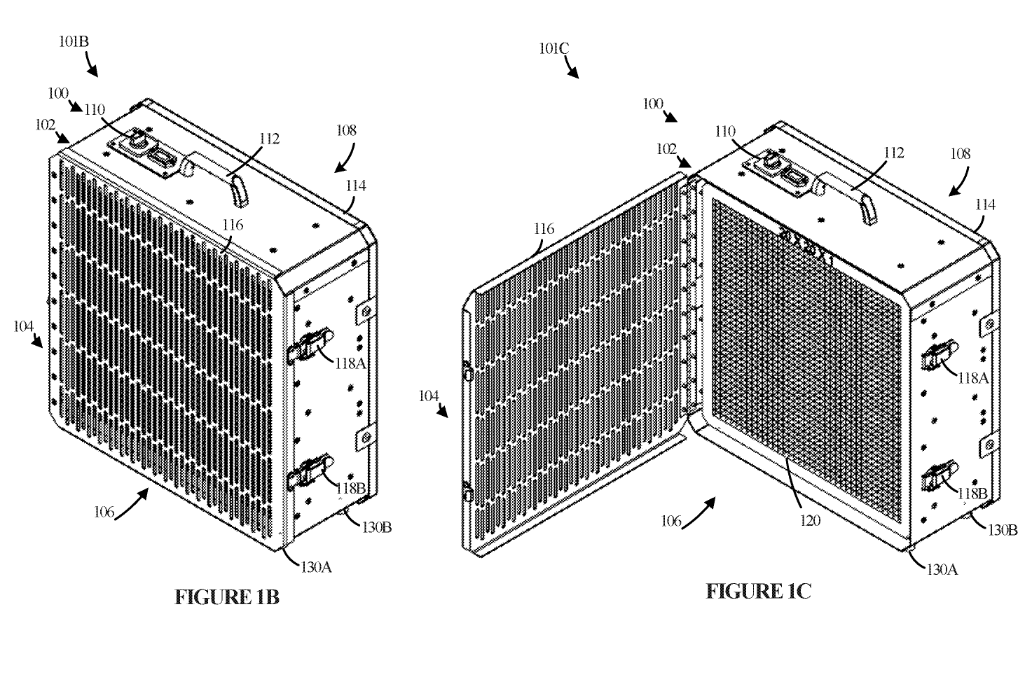

Turning now to the Figures, FIG. 1A illustrates an isometric perspective 101A of a portable ozone generator 100 according to an embodiment herein. As shown, the portable ozone generator 100 includes a portable ozone generator housing 102 that defines and supports a housing opening 104 extending between an air intake side 106 and an air discharge side 108. The housing 102 further supports a control panel 110, a handle 112, a rear door 114, a front access door in the form of a hinged door 116, latches 118A and 118B, a filter 120, an ozone generation module 122, a fan 124, fan brackets 128, and housing feet 130A and 130B.

In the illustrated example, the housing 102 is a rigid, transportable enclosure configured to protect and support the internal mechanical and electrical components of the device. The housing opening 104 defines a central airflow passage extending between the air intake side 106 and the air discharge side 108. The air intake side 106 establishes the structural boundary through which ambient air is drawn into the portable ozone generator 100, while the air discharge side 108 defines the structural boundary through which ozone-containing air is expelled into a treatment zone. The housing 102 further provides mounting structures for the handle 112, the control panel 110, the latches 118A and 118B, the filter 120, and the ozone generation module 122, and includes internal support features for the fan 124, the fan brackets 128, and the housing feet 130A and 130B.

In some embodiments, the portable ozone generator 100 may have dimensions and a mass configured for manual transport, including a width between approximately 15–35 inches, a height between approximately 15–35 inches, a depth between approximately 5–15 inches, and a mass up to approximately 50 pounds. In other embodiments, the portable ozone generator 100 has a mass greater than 50 pounds, such as between 50 and 100 pounds. The portable ozone generator 100 may be “portable” such that it can be lifted, carried, repositioned, or otherwise transported by a user without the need for mechanical assistance, wheeled carts, powered lifting equipment, or multiple operators. The handle 112 may be positioned and reinforced to support balanced manual lifting, and the overall distribution of mass within the housing 102 may be arranged to facilitate safe, single-person transport. Other embodiments may incorporate lighter-weight constructions having masses below approximately 40 pounds, below approximately 30 pounds, or even below approximately 25 pounds for enhanced portability, while larger embodiments may preserve manual-transport capability through ergonomic handle placement, reduced-profile housing geometries, or weight-reducing material selections.

In some embodiments, the control panel 110 includes user-operable controls such as fan-speed selectors, run-timers, power switches, and indicator elements. The handle 112 provides a reinforced lifting point for transporting the ozone generator 100. The rear door 114 forms a rear enclosure surface and may be hinged or removable to permit maintenance access. The hinged door 116, positioned on the air intake side 106, is one example of a front access door configuration. Although a hinged door is illustrated, other door architectures—including removable panels, sliding access covers, latched or gasketed front plates, or quick-release service doors—are contemplated herein. The latches 118A and 118B secure the hinged door 116 in a closed configuration during operation. The filter 120 is positioned immediately behind the hinged door 116 at the air-entry region and may include various filtration media depending on the intended environment.

The ozone generation module 122 includes one or more discrete ozone generation units 122A–122J. Each ozone generation unit may be or include a corona-discharge ozone-producing element configured to convert molecular oxygen (O₂) in the incoming airstream into ozone (O₃). In the illustrated embodiment, each ozone generation unit 122A–122J includes dielectric layers, electrode surfaces, and associated high-voltage structures arranged to generate localized corona discharge when energized. The ozone generation units 122A–122J are positioned around a perimeter of the housing opening 104, forming an annular ring surrounding the central airflow passage. This perimeter-mounted configuration provides several technical advantages: (i) it maximizes the exposure of incoming air to the active corona-discharge surfaces without obstructing the high-velocity airflow produced by the fan 124; (ii) it prevents airflow stagnation that typically occurs in the low-velocity region near the fan motor hub, thereby maintaining oxygen availability for ozone conversion; (iii) it distributes ozone generation evenly around the intake region, promoting uniform mixing of ozone into the air stream; and (iv) it improves thermal dissipation and electrical load distribution by spreading the corona-discharge components around the perimeter of the housing. In some embodiments, the ozone generation units 122A–122J are arranged equidistantly to maintain balanced electrical loading, symmetric thermal distribution, and uniform airflow interaction within the ozone generation module 122.

In other embodiments, however, the ozone generation units 122A–122J may be arranged in a variety of alternative spatial configurations depending on desired ozone output, airflow characteristics, electrical-routing considerations, or enclosure geometry. For example, the ozone generation units may be positioned non-equidistantly around the housing opening 104 to accommodate wiring paths, thermal-management structures, or localized airflow zones. In some embodiments, the ozone generation units may be clustered in groups or segments, arranged in staggered or offset patterns, or distributed among multiple concentric rows to increase corona-discharge surface area or create higher output variants. In still other embodiments, the ozone generation units may be arranged in a partial ring configuration that occupies only a subset of the perimeter, or may be distributed on multiple interior surfaces of the housing 102, including upstream or downstream planes relative to the fan 124. Additional embodiments may position ozone generation units on both the intake and discharge sides of the housing opening 104, or integrate auxiliary ozone-generation elements in sidewalls, internal brackets, or modular cartridge assemblies. These configuration variations enable the ozone generation module 122 to be adapted for different airflow patterns, electrical loading schemes, thermal behavior, or ozone-production capacities while operating according to the structural and functional principles described herein.

In some embodiments, one or more of the ozone generation units 122A–122J are removably mounted within the ozone generation module 122 to permit individual replacement, exchange, or reconfiguration. For example, a given ozone generation unit 122A–122J may be affixed within the ozone generation module 122, or to the housing 102, in a manner that allows the unit to be removed independently of the other ozone generation units. Such mounting arrangements may include mechanical fasteners such as screws, bolts, pins, clips, clamps, frames, rails, or snap-fit structures, as well as removable cartridge housings or bracket assemblies configured to retain the ozone generation unit in a defined position during operation. In some embodiments, electrical connections to each ozone generation unit 122A–122J may be provided through plug-in connectors, contact pads, spring contacts, or other separable electrical interfaces, enabling a unit to be removed and replaced without rewiring the ozone generation module 122. This replaceable configuration facilitates servicing, allows substitution of ozone generation units having different output ratings or electrical characteristics, and supports modular scaling of ozone production capacity over the operational life of the portable ozone generator 100.

Although the illustrated embodiment includes ten ozone generation units 122A–122J arranged around the perimeter of the housing opening 104, other embodiments may include more or fewer ozone generation units depending on desired ozone production capacity, electrical-load constraints, enclosure size, airflow rate, or target treatment-zone volume. For example, smaller implementations intended for residential or low-volume applications may include as few as two to four ozone generation units, while larger or industrial-grade variants may incorporate twelve, sixteen, or more ozone generation units positioned in a ring, segmented array, or multi-row configuration. Similarly, the ozone generation units may vary in output rating—such as 10 g/hr, 20 g/hr, 30 g/hr, or higher—and may be selected, mixed, or sequenced to achieve a desired aggregate output. This modular architecture enables the ozone generation module 122 to be scaled to different power envelopes, treatment requirements, or airflow characteristics without departing from the core structural and functional principles described herein.

The fan 124 is a high-volume air-moving assembly positioned centrally within the housing opening 104. In the illustrated embodiment, the fan 124 is an axial-flow fan that generates substantial volumetric airflow through the portable ozone generator 100, enabling high-rate air transport across the ozone generation module 122 and into a treatment zone. The fan 124 may include multiple blades formed from metal or high-strength polymer materials selected for rigidity, aerodynamic efficiency, and resistance to ozone exposure. The fan 124 is driven by an electric motor configured to provide sufficient torque to sustain airflow rates appropriate for ambient-air-fed ozone mass transfer. High-volume airflow produced by the fan 124 helps avoid localized oxygen depletion around the ozone generation units 122A–122J, prevents formation of stagnant ozone pockets, and enhances mixing of ozone with the incoming air stream.

It should be appreciated that while the present discussion focuses on embodiments leveraging a bladed fan, such as the fan 124, for moving air through the housing, in some cases, a bladeless fan may be used. In some embodiments, the portable ozone generator 100 may employ a bladeless air-moving device configured to induce or amplify airflow through the housing opening 104 without the use of rotating fan blades positioned directly in the airflow path.

For example, the air-moving device may include a bladeless airflow generator that utilizes a pressurized air track, air-amplification channel, annular slot, or internal flow-guidance structure to entrain and accelerate ambient air through the opening. Such systems may operate by projecting a high-velocity air stream along a curved or contoured surface to induce additional airflow via entrainment and pressure differentials, thereby generating a net volumetric flow comparable to that produced by a conventional bladed fan. In these embodiments, the induced airflow may perform the same functional role as the bladed fan described above (i.e., drawing ambient air into the air intake side, propelling the air across the ozone generation module arranged around the perimeter of the opening, and discharging ozone-containing air into the treatment zone).

The fan brackets 128 secure the fan 124 within the housing opening 104 in a fixed and precisely aligned orientation relative to the airflow path. The fan brackets 128 may include vibration-resistant mounting features, reinforcing ribs, or cross-members that ensure stable fan operation during transport and ozone-generation cycles. Proper alignment reduces turbulence, maintains uniform airflow, and supports consistent ozone production efficiency across the ozone generation module 122. The housing feet 130A and 130B provide stable contact surfaces when the portable ozone generator 100 is placed on a floor or other support structure and may include elastomeric or vibration-dampening features to reduce operational movement or noise.

In some embodiments, the portable ozone generator 100 may be powered using a variety of electrical power sources suitable for field, commercial, or residential operation. For example, the portable ozone generator 100 may operate on standard alternating-current (AC) electrical service, including approximately 120-volt residential circuits or 208–240-volt commercial or industrial circuits, and may draw less than approximately 15 amps during operation. In other embodiments, the portable ozone generator 100 may be powered by direct-current (DC) sources, such as portable battery packs, external battery modules, or vehicle-supplied power systems, enabling treatment in remote locations without access to fixed electrical infrastructure. Additional embodiments may utilize portable power generators, inverter-based supplies, or dual-mode AC/DC architectures that permit automatic or user-selected switching between power sources. The power entry region of the housing 102 may include protective conduit structures, such as astrain relief cord connector, strain-relief fittings, or sealed electrical interfaces, to ensure safe and reliable electrical connectivity regardless of the selected power configuration.

FIG. 1B illustrates a closed-door perspective 101B of the portable ozone generator 100, according to an embodiment herein. In this configuration, the hinged door 116 is secured in the closed position by the latches 118A and 118B, forming a continuous exterior surface on the air intake side 106. The filter 120 is positioned directly behind the hinged door 116, while the ozone generation units 122A–122J remain arranged around the perimeter of the housing opening 104. The fan 124 is centrally mounted within the housing opening 104 using the fan brackets 128. The housing feet 130A and 130B provide stable ground support for the unit.

When the hinged door 116 is closed, the door 116 protects internal components—including the filter 120 and the ozone generation units 122A–122J—from accidental contact, airborne debris, or external contamination. The closed door 116, together with the air intake side 106 and the air discharge side 108, establishes the exterior airflow boundaries of the housing 102. Although a hinged door is depicted, alternate front-access configurations capable of providing equivalent protection and serviceability are also suitable.

FIG. 1C illustrates an open-door perspective 101C of the portable ozone generator 100, according to an embodiment herein. In this view, the hinged door 116 pivots away from the air intake side 106 while remaining attached to the housing 102 via the hinge structures. Opening the hinged door 116 exposes the filter 120, permitting inspection, cleaning, and replacement. With the filter 120 removed, the plurality of ozone generation units 122A–122J arranged around the perimeter of the housing opening 104 becomes accessible for maintenance. The fan 124 remains centrally mounted in the housing opening 104, supported by the fan brackets 128, and the housing feet 130A and 130B maintain stability during servicing.

In the open configuration, the hinged door 116 provides clear access to the internal air-entry region, enabling maintenance of the filter 120, cleaning of the ozone generation units 122A–122J, and inspection of the internal airflow path. Other access-panel configurations may also be employed to provide similar levels of serviceability without departing from the scope of the embodiments described herein.

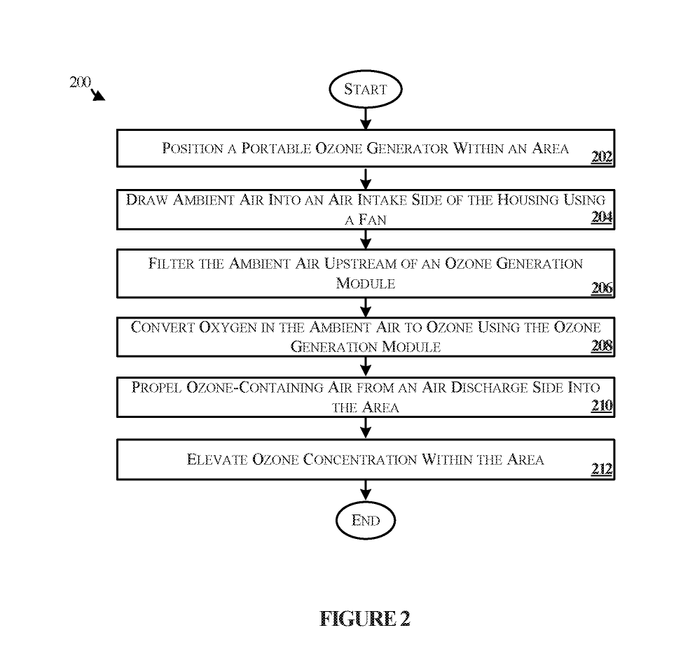

Referring now to FIG. 2, a method 200 is illustrated for performing a biocidal air and surface treatment using the portable ozone generator 100 of FIGS. 1A–1C. Although method 200 is described using the portable ozone generator 100 as an example, the steps of method 200 may be applied to other ozone-generation systems described herein, including systems having alternative housing configurations, different airflow capacities, or different numbers and arrangements of ozone generation units.

The method 200 begins by positioning the portable ozone generator 100 within an area selected for treatment (Step 202). The portable ozone generator 100 may be placed on a floor surface, an elevated platform, or a suspended mount depending on the geometry and intended airflow distribution within the treatment zone. In some embodiments, the portable ozone generator 100 is positioned centrally to promote radial ozone dispersion, while in other embodiments, the portable ozone generator 100 may be oriented toward high-traffic or high-contamination regions where rapid ozone buildup is desired. In larger structures such as agricultural barns, warehouses, or industrial spaces, multiple portable ozone generators 100 may be positioned in a distributed pattern to generate overlapping ozone fields and achieve uniform treatment across expansive or irregularly shaped environments.

Once positioned and initiated, the portable ozone generator 100 draws ambient air into the air intake side 106 as part of the operational sequence (Step 204). The fan 124 induces airflow through the housing opening 104 at volumetric flow rates that may range from approximately from 1,500 to 5,500 standard cubic feet per minute (SCFM), from 1,800 to 4,600 SCFM, or from 2,200 to 4,000 SCFM, depending on fan speed, blade geometry, and system settings. Alternative embodiments may employ higher-capacity fans capable of exceeding 5,000 SCFM or lower-capacity fans configured for residential or smaller commercial spaces. This intake airflow establishes a continuous supply of oxygen-containing air that is necessary for ozone generation and prevents stagnant air pockets that could otherwise reduce ozone conversion efficiency. In some embodiments, airflow may be manually selected using the control panel 110, or automatically modulated based on sensed ozone concentration, humidity, occupancy schedules, or a programmed treatment profile.

As ambient air enters the portable ozone generator 100, the air is filtered by the filter 120 (Step 206). The filter 120 removes particulates, dust, organic debris, and other airborne contaminants that could accumulate on the ozone generation units 122A–122J or interfere with corona-discharge stability. Depending on the deployment environment, the filter 120 may include a mesh pre-filter, a MERV-rated filter, an electrostatic filter, or in some embodiments a HEPA-grade filter for fine particulate retention. In environments with high particulate density—such as poultry barns, livestock enclosures, grain storage facilities, or industrial workshops—multiple filtration stages or more frequent filter replacement may be employed. Certain embodiments may incorporate airflow sensors or filter-load indicators to alert an operator when filter replacement is required to maintain ozone generation efficiency.

After filtration, the conditioned air flows across the ozone generation module 122122, where the portable ozone generator 100 converts oxygen to ozone (Step 208). As described with respect to FIGS. 1A–1C, the ozone generation module 122 may include multiple ozone generation units 122A–122J arranged around a perimeter of the housing opening 104. Each ozone generation unit is a corona-discharge element configured to dissociate molecular oxygen into atomic oxygen, which subsequently recombines into ozone. As air flows across these electrically energized surfaces, ozone is produced at aggregate rates ranging in some embodiments from approximately 50 g/hr to 450 g/hr, , from 60 g/hr to 385 g/hr, 70 g/hr to 360 g/hr, or from 100 g/hr to 300 g/hr, although higher or lower production rates may be used depending on the number, size, energy level, and arrangement of ozone generation units. The perimeter-mounted configuration of the ozone generation units 122A–122J increases the effective interaction surface area of corona-discharge elements with the incoming air stream, preserves high-velocity airflow through the central region of the housing opening 104, avoids oxygen-depleted stagnation zones near the fan hub, and provides more uniform thermal and electrical loading across the ozone generation module 122.

The ozone-containing air is then propelled outward through the air discharge side 108 by the fan 124 (Step 210). The fan 124 generates a high-velocity airflow stream that projects ozone-laden air into the surrounding treatment zone, promoting rapid mixing and distribution. Depending on airflow rate, discharge geometry, and environmental obstructions, ozone may disperse radially, follow linear flow paths, or develop circulating convection patterns. In certain embodiments, the portable ozone generator 100 may incorporate directional louvers, oscillating discharge mechanisms, or adjustable airflow guides to steer ozone flow within complex room geometries. When multiple portable ozone generators 100 are employed, the airflow patterns generated by each portable ozone generator 100 may be arranged to complement one another, promoting cross-ventilation or synchronized plume propagation for faster homogenization of ozone concentration.

As the ozone-containing air mixes throughout the treatment zone, ozone concentration rises toward a biocidal range suitable for air and surface disinfection (Step 212). In many embodiments, the portable ozone generator 100 elevates ozone concentration within a range of approximately 2 to 100 PPM, from 4 to 70 PPM, from 10 to 60 PPM, or from 25 to 75 PPM within a period of about 10 to 90 minutes, in some cases, 15 to 60 minutes, or even 15 to 45 minutes, though longer or shorter durations may be used depending on room volume, environmental conditions, airflow settings, or target disinfection requirements. Environmental factors such as humidity, temperature, and baseline air exchange rates may influence concentration buildup and may be considered when selecting operational parameters. In some embodiments, ozone concentration is monitored using a handheld detector, an onboard ozone sensor, or a distributed sensing network. Treatment may continue until a required concentration-time product is achieved, after which the portable ozone generator 100 may automatically shut down, transition to a standby mode, or initiate a post-treatment decay cycle.

In some embodiments, the portable ozone generator 100 includes an onboard ozone concentration sensor positioned at or near the air discharge side 108, within the housing opening 104, or on the control panel 110. The ozone concentration sensor is configured to measure ozone levels within the treatment zone and to provide real‑time concentration data to a control system associated with the control panel 110. Depending on the application, the ozone concentration sensor may be an electrochemical sensor, UV absorption sensor, semiconductor-based sensor, or any other ozone-detecting technology. The ozone concentration sensor may be mounted internally, externally, in the airstream, or remotely and communicate wirelessly with the control panel 110

Based on the detected ozone concentration, the portable ozone generator 100 may automatically modulate the operational state of the fan 124 to regulate ozone distribution and maintain a desired concentration profile. For example, when the ozone concentration sensor detects that the ozone level is below a target threshold, the control system may increase the fan 124 speed to elevate airflow and accelerate dispersion of ozone‑containing air into the treatment zone. Conversely, if the detected ozone concentration exceeds a threshold value or approaches a predetermined upper limit, the control system may reduce the fan 124 speed, cycle the fan 124 into a lower‑energy mode, or temporarily deactivate ozone production while maintaining minimal airflow for circulation. The control panel 110 may further permit an operator to set concentration thresholds, adjust modulation sensitivity, or select among multiple automated control modes tailored for residential, commercial, or industrial treatment scenarios. This closed‑loop feedback configuration enables the portable ozone generator 100 to adjust airflow output to optimize ozone distribution, prevent localized over‑concentration, and maintain stable operational performance within a wide range of environmental conditions.

In embodiments where the portable ozone generator 100 is part of a coordinated multi‑unit system, multiple portable ozone generators 100 may communicate wirelessly to adjust output levels, synchronize operation across zones, or maintain uniform ozone concentration throughout a treatment zone. In such embodiments, each portable ozone generator 100 may monitor local operating conditions—such as fan status, elapsed treatment time, or sensed environmental parameters—and exchange this information with other units via a wireless communication module on the control panel 110. The coordinated control logic may include adjusting fan 124 speed, staging or sequencing ozone generation units 122A–122J on or off, or synchronizing treatment intervals so that ozone concentration rises uniformly across the facility rather than peaking unevenly near individual devices. When deployed in large agricultural, commercial, or industrial environments, this coordinated multi‑unit operation helps distribute ozone more evenly, minimizes untreated pockets within the treatment zone, and maintains a balanced concentration profile during the entire treatment cycle.

Following treatment, additional steps may include permitting natural ozone decay, activating carbon-based ozone scrubbers, ventilating the space, or verifying ozone dissipation prior to reentry by personnel.

As noted above, in some embodiments, the portable ozone generator 100 may operate as part of a coordinated, automated, or semi-automated ozone-delivery system that includes one or more additional portable ozone generators 100 deployed within the same treatment zone or across multiple adjacent zones. These system-level embodiments enable enhanced performance, improved distribution uniformity, safety control, and intelligent modulation of ozone output beyond the capabilities of a single manually operated unit.

In one embodiment, the portable ozone generator 100 includes an onboard control system configured to monitor ozone concentration, manage fan 124 speed, sequence the activation of ozone generation units 122A–122J, and execute time-based or concentration-based control routines. The control system may receive ozone-concentration data from an onboard ozone sensor mounted on or near the air discharge side 108, within the housing opening 104, or integrated into the control panel 110. Based on the detected ozone level, the control system may automatically increase fan 124 speed, activate additional ozone generation units 122A–122J, modulate the duty cycle of the ozone generation module 122, or temporarily suspend ozone output if a threshold concentration is reached. Such closed-loop operation enables the portable ozone generator 100 to maintain a target ozone concentration profile without continuous user intervention.

In some embodiments, multiple portable ozone generators 100 deployed within a facility may communicate wirelessly to coordinate their operation. Each portable ozone generator 100 may include a wireless communication module integrated into the control panel 110 and configured to support communication via Bluetooth®, Wi-Fi®, or a local-area-network protocol. Through this wireless communication link, the portable ozone generators 100 can exchange operational data such as fan speed, ozone concentration, treatment duration, airflow mode, or status information. A distributed coordination algorithm may utilize this shared data to synchronize treatment cycles, balance ozone output among devices, or maintain a uniform ozone concentration across a large or irregular treatment zone.

For example, if a first portable ozone generator 100 detects that ozone concentration near its discharge side 108 is increasing more rapidly than in other areas, the device may automatically reduce its output or fan speed, while signaling neighboring portable ozone generators 100 to increase their respective outputs. Conversely, if a sensor associated with another portable ozone generator 100 indicates that ozone concentration remains below a target threshold, one or more portable ozone generators 100 may increase ozone generation or airflow to ensure that target concentrations are achieved uniformly across the entire treatment zone. This coordinated operation enables rapid homogenization of ozone levels in large facilities, improves biocidal treatment consistency, and reduces the likelihood of localized over-concentration or under-treatment.

In some embodiments, the wireless communication module enables remote operation of the portable ozone generator 100 from outside the treatment zone. An operator may use a remote controller, smartphone application, tablet interface, or building-automation system to initiate or terminate treatment cycles, adjust fan 124 speed, modify ozone-generation intensity, monitor real-time ozone concentrations, or configure automated treatment schedules. Remote operation is advantageous in scenarios where ozone concentration rises to levels unsuitable for human occupancy, allowing operators to remain outside the treatment zone while maintaining full control over the treatment sequence.

In further embodiments, system-level coordination may include hierarchical control architectures, such as a designated “primary” portable ozone generator 100 that issues synchronization commands to a plurality of secondary units, or fully distributed peer-to-peer coordination in which each portable ozone generator 100 autonomously adjusts its operation based on data received from neighboring units. Additional implementations may incorporate distributed sensing networks positioned throughout the facility, enabling more granular ozone-concentration monitoring and more precise modulation of device output. Such sensing networks may include fixed ozone sensors, mobile sensors, or sensors integrated into existing HVAC infrastructure.

Collectively, these system-level coordination and automation features enable the portable ozone generator 100 to operate in complex environments, large-scale facilities, and multi-room structures with improved safety, efficiency, and treatment uniformity. These embodiments provide flexibility for single-unit operation, multi-unit distributed operation, and remote operator oversight, allowing the technology to be adapted to residential, commercial, agricultural, and industrial use cases.

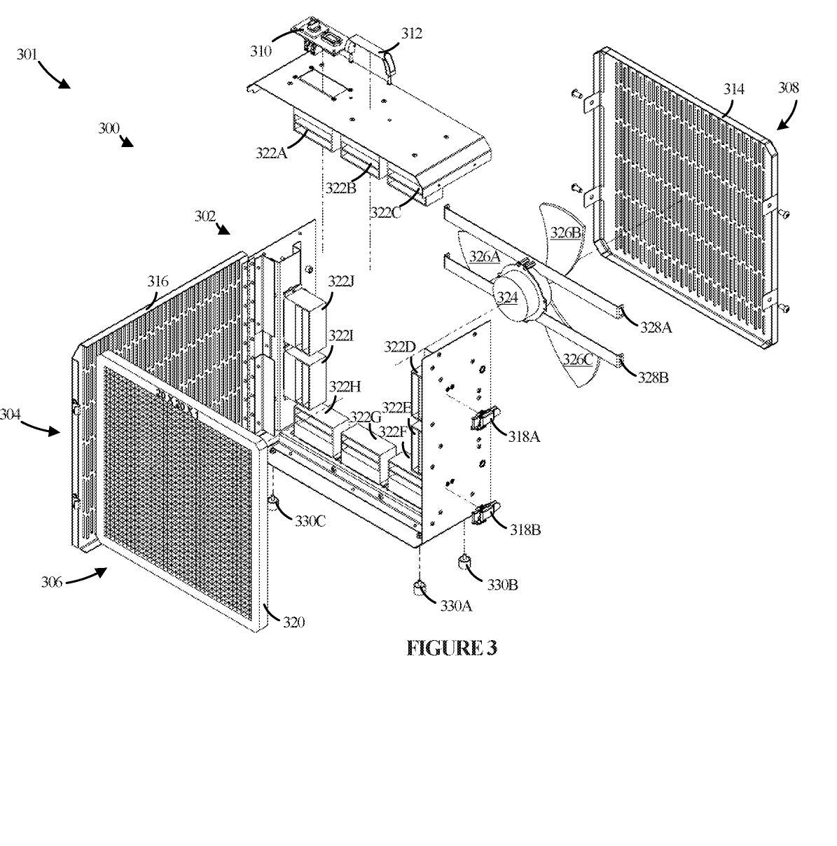

Referring now to FIG. 3 which illustrates a blow-out perspective 301 of a portable ozone generator 300, according to an embodiment herein. The portable ozone generator 300 may be the same as, or structurally and functionally similar to, the portable ozone generator 100 described above with respect to FIGS. 1A–1C. Accordingly, only those features specifically illustrated in FIG. 3 or differing from the previous embodiment are described in detail below.

The portable ozone generator 300 includes a portable ozone generator housing 302 that defines a housing opening 304 extending between an air intake side 306 and an air discharge side 308. In the illustrated embodiment, the housing 302 supports components similar to those described above, including a filter 320 at the air intake side 306, a handle 312, a rear door 314, and a front access door in the form of a hinged door 316 secured by latches 318A and 318B. Housing feet 330A, 330B, and 330C provide ground support and may include vibration-dampening features.

In some embodiments, the portable ozone generator 300 may have dimensions and a mass configured for manual transport. For example, the housing 302 may have a width between approximately 15 inches and 35 inches, a height between approximately 15 inches and 35 inches, and a depth between approximately 5 inches and 15 inches. In certain implementations, the portable ozone generator 300 may have a mass of up to approximately 50 pounds to enable manual lifting, carrying, and repositioning during field deployment. Other embodiments may utilize alternative manually portable dimensional ranges—such as widths between approximately 12 and 40 inches, heights between approximately 12 and 40 inches, depths between approximately 4 and 20 inches, or masses below approximately 40 pounds or even 25 pounds—depending on the selected fan diameter, ozone-generation capacity, enclosure materials, or intended use case. These dimensional characteristics may be similar to, or scaled variants of, those described with respect to the portable ozone generator 100 in FIGS. 1A–1C

The portable ozone generator 300 includes a control panel 310 that may have the same or similar operational controls as the control panel 110 described with respect to FIGS. 1A–1C. The control panel 310 may include user-operable fan controls, ozone-output selectors, run-time settings, operational indicators, and safety interlocks. In some embodiments, the control panel 310 includes a wireless communication module that enables remote operation of the portable ozone generator 300 from outside the treatment zone, allowing an operator to initiate or terminate a treatment cycle, adjust fan speed, or monitor real-time ozone concentration without entering the treated space. The wireless communication module may further support coordinated, multi-unit operation in scenarios where multiple portable ozone generators 300 are deployed in a facility. In such embodiments, multiple portable ozone generators 300 may exchange operational data and dynamically adjust their output levels, synchronize treatment intervals, or cooperatively maintain a uniform ozone concentration across a large or irregular treatment zone.

The portable ozone generator 300 further includes an ozone generation module 322 comprising a plurality of discrete ozone generation units 322A–322J. Each ozone generation unit may include dielectric plates, electrode structures, and high-voltage discharge elements configured to generate localized corona discharge for converting oxygen to ozone. While the illustrated embodiment depicts ten ozone generation units, other embodiments may include additional or fewer units depending on desired ozone output, power constraints, airflow characteristics, or enclosure geometry. For example, smaller implementations may include as few as two to four ozone generation units, while higher-capacity industrial models may incorporate twelve, sixteen, or more units arranged in one or more rows around the housing opening 304. Collectively, the ozone generation units 322A–322J form the ozone generation module 322 and are positioned around a perimeter of the housing opening 304, forming an annular ring surrounding the central airflow path. This perimeter-mounted configuration maximizes the exposure of incoming air to the active corona-discharge surfaces, avoids the low-velocity region near the central fan hub, promotes uniform ozone mixing within the airstream, and distributes electrical and thermal loading more evenly across the ozone generation module 322.

The fan 324 is positioned centrally within the housing opening 304 and may be similar to the fan 124 described above. In some embodiments, the fan 324 is a multi-speed axial-flow fan configured to move air at volumetric flow rates ranging from approximately 1,800 to 4,600 cubic feet per minute. The fan blades 326A, 326B, and 326C may be fabricated from a variety of durable, ozone-resistant materials, including aluminum, stainless steel, galvanized steel, anodized aluminum alloys, high-strength polymer composites such as glass-filled nylon or fiberglass-reinforced plastic, carbon-fiber composite materials, or other engineered polymers formulated to withstand prolonged exposure to ozone. In some implementations, the fan blades may include protective surface treatments—notably powder coating, anodizing, polymer over-molding, or corrosion-resistant plating—to enhance rigidity and durability in high-ozone environments. The fan blades 326A–326C may further be manufactured with selected pitch angles, airfoil geometries, swept profiles, or aerodynamic shaping features to optimize volumetric airflow, minimize turbulence, reduce operational noise, and improve airflow uniformity across the ozone generation module 322. The fan brackets 328A and 328B secure the fan 324 in a fixed, aligned orientation relative to the housing opening 304 to ensure uniform airflow across the ozone generation units 322A–322J.

During operation, the portable ozone generator 300 may elevate ozone concentration within a treatment zone to a biocidal range between approximately 4 and 70 parts per million (PPM). In some embodiments, the treatment zone comprises an enclosed area having a volume of up to approximately 10,000 cubic feet for a single portable ozone generator 300. Because the portable ozone generator 300 relies solely on ambient air as the oxygen source, the maximum achievable ozone concentration is inherently limited by the available oxygen content of the incoming air. This ambient‑air‑limited behavior allows the portable ozone generator 300 to achieve ozone concentrations sufficient for microbial inactivation while avoiding the extreme, material‑damaging ozone levels characteristic of oxygen‑fed industrial systems.

In some embodiments, the portable ozone generator 300 elevates ozone concentration within the treatment zone to the biocidal range over a treatment duration selected based on environmental conditions, airflow characteristics, and ozone generation module configuration. For example, ozone concentrations within the treatment zone may be achieved within approximately 15 to 45 minutes, although shorter or longer durations may be used depending on room volume, baseline air exchange, humidity, temperature, or selected fan operating parameters. The separation of concentration level and exposure time enables flexible treatment protocols tailored to specific applications and microbial targets.

In further embodiments, the treatment zone may exceed approximately 10,000 cubic feet. Larger treatment zones may be addressed by modifying system configuration, increasing airflow capacity, increasing aggregate ozone generation output, or deploying multiple portable ozone generators 300 within the same facility. In multi‑unit deployments, the portable ozone generators 300 may be positioned to create overlapping ozone fields and uniform distribution across large or irregular spaces such as barns, warehouses, manufacturing floors, or multi‑room structures. Under such configurations, the effective treatment zone size scales with the number of portable ozone generators 300, their placement, and their coordinated operation, while still maintaining ambient‑air‑limited ozone concentration behavior..

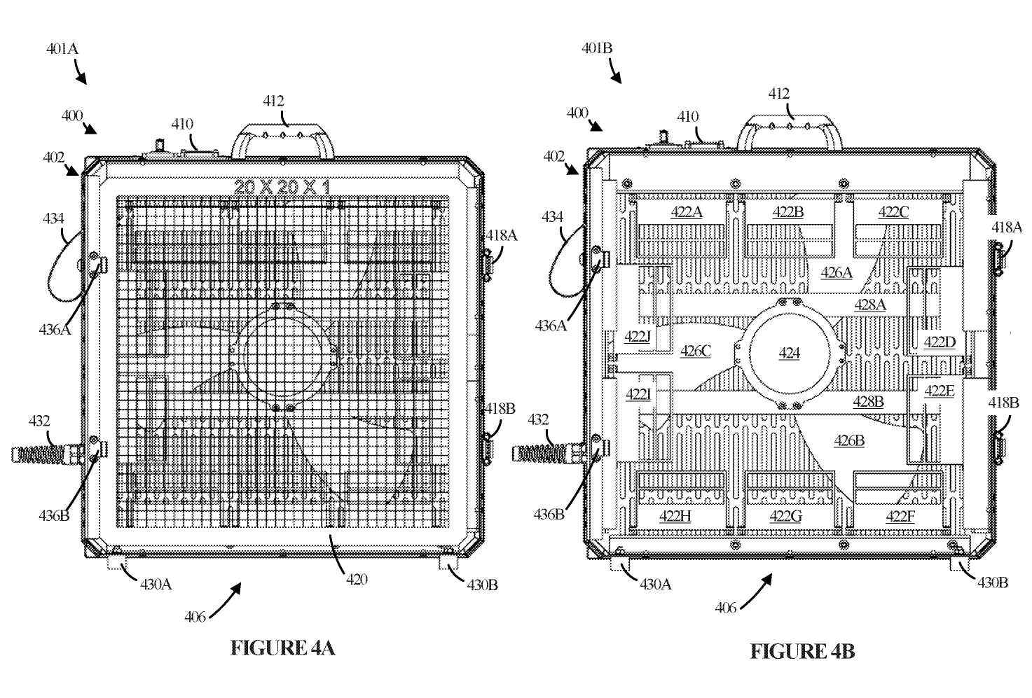

Referring now to FIGS. 4A and 4B, perspectives views 401A and 401B, respectively, of a portable ozone generator 400 are provided, according to an embodiment herein. The portable ozone generator 400 may be the same as, or structurally and functionally similar to, the portable ozone generator 100 described with respect to FIGS. 1A–1C, and therefore only those features specifically illustrated in FIGS. 4A and 4B or differing from the earlier embodiments are described in detail below.

In FIG. 4A, the portable ozone generator 400 is shown with its front access door removed to expose the air intake side 406 and the filter 420 positioned at the air-entry region. The housing 402 supports a control panel 410 and a handle 412 on its upper surface, and latches 418A and 418B remain mounted to the front frame of the housing 402 to indicate their alignment with the removed door. Hinges 436A and 436B are positioned along one vertical side of the housing 402, identifying the hinge attachment location for the front access door in normal operation. A strain relief cord connector 432 is shown coupled along one side of the housing 402 to protect the incoming power cord from bending or fatigue and to guide the cord away from the generator during transport or operation. Housing feet 430A and 430B are mounted to the underside of the housing 402 and provide stable ground support during positioning and use.

With the front access door removed, FIG. 4A illustrates the serviceable intake region of the portable ozone generator 400, including the placement of the filter 420 within the air intake side 406. This configuration highlights how the filter 420 can be accessed, removed, cleaned, or replaced during routine maintenance and demonstrates the alignment of the latch structures 418A–418B and hinges 436A–436B that secure the front access door when installed. The illustrated arrangement supports ease of service, airflow entry, and safe access to components positioned directly behind the intake panel.

FIG. 4B illustrates the portable ozone generator 400 with both the front access door and the filter 420 removed, providing a view into the internal intake-side configuration. With the filter removed, the plurality of ozone generation units 422A–422J are visible, arranged around a perimeter of the intake opening in a ring-type configuration similar to the arrangement of ozone generation units described previously with respect to FIGS. 1A–1C and 3. The ozone generation units 422A–422J are positioned behind the filter location such that incoming air passes first through the filter 420 (when installed) and then across the active corona-discharge surfaces of the ozone generation units. Fan brackets 428A and 428B are also visible behind the intake region and provide structural support for the centrally mounted fan, which draws air through the intake side 406 and propels the air toward the discharge side.

By exposing the intake interior, FIG. 4B demonstrates how the intake region is structured to receive a high-volume airflow from the fan once the filter 420 and front access door are reinstalled. The figure also illustrates the spatial relationship between the filter location, the perimeter-mounted ozone generation units 422A–422J, and the fan brackets 428A–428B. The arrangement of hinges 436A–436B, latches 418A–418B, and the strain relief cord connector 432 further shows how these supporting components interface with the housing 402 during normal operation, maintenance, and transport.

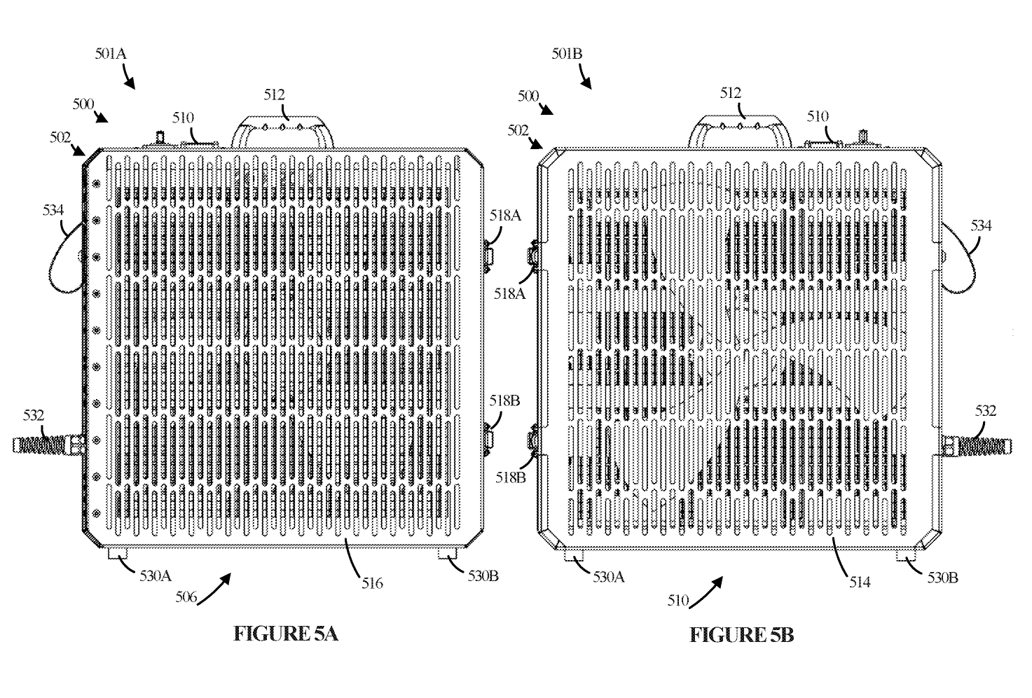

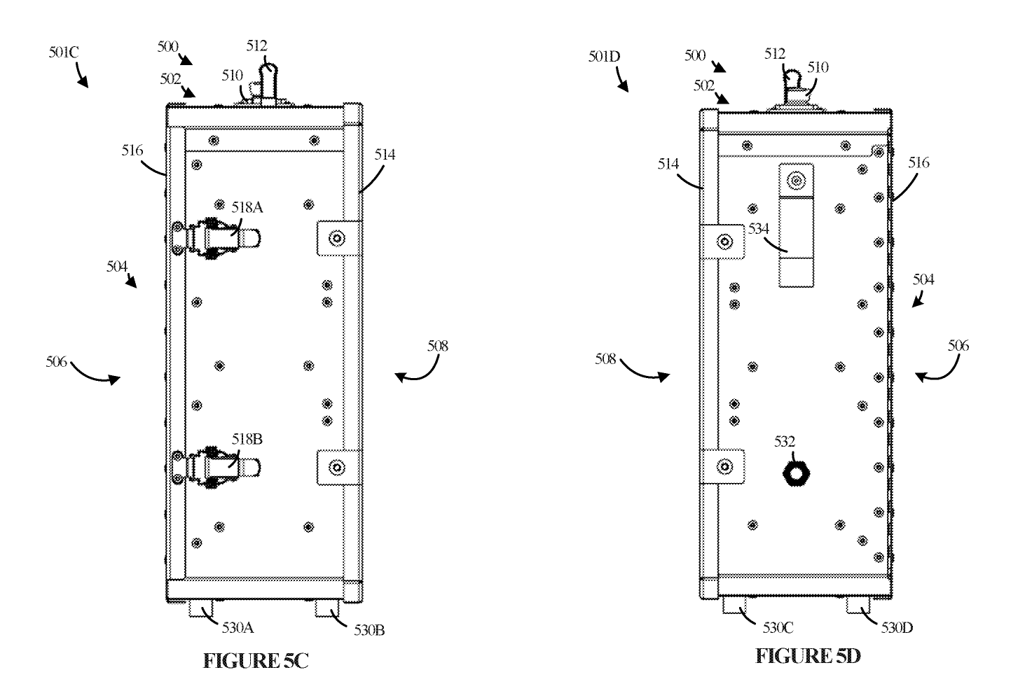

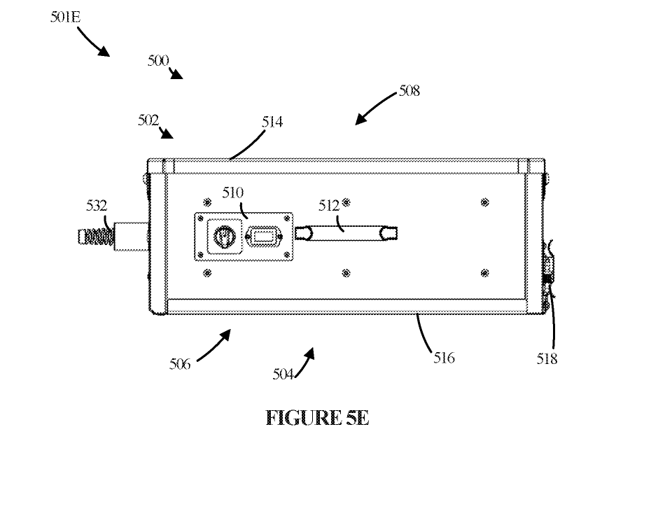

Referring now to FIGS. 5A-5E, perspective views 501A–501E of a portable ozone generator 500 are provided, according to an embodiment herein. The portable ozone generator 500 may be the same as, or structurally and functionally similar to, the portable ozone generator 100 described with respect to FIGS. 1A–1C. Accordingly, only those features specifically illustrated in FIGS. 5A–5E, or features that differ from earlier embodiments, are described in detail below.

FIG. 5A illustrates a front-facing perspective 501A of the portable ozone generator 500 with the hinged door 516 in the closed position. The portable ozone generator 500 includes a portable ozone generator housing 502, a housing opening 504, an air intake side 506, an air discharge side 508, a control panel 510, a handle 512, latches 518A and 518B, housing feet 530A and 530B, a strain relief cord connector 532, and a hinge 534. As shown, the hinged door 516 covers the filter location and completes the intake surface of the air intake side 506, while the latches 518A and 518B secure the door in place during operation. The strain relief cord connector 532 provides strain relief and guides the electrical cord away from the housing 502, and the control panel 510 and handle 512 are mounted along the upper portion of the enclosure.

FIG. 5B illustrates a rear-facing perspective 501B of the portable ozone generator 500. The rear door 514 encloses the back side of the housing 502 and provides service access to interior components when opened. The latches 518A and 518B remain positioned along the intake side 506 to interface with the hinged door 516 on the opposite face, while the strain relief cord connector 532 and housing feet 530A and 530B maintain the electrical-entry and ground-support functionality described above. This view highlights the overall enclosure geometry and external component placement from a rear vantage point.

FIG. 5C illustrates a side-facing perspective 501C of the portable ozone generator 500 with the hinged door 516 in the closed and latched position. The housing 502 supports the housing opening 504, the handle 512, the control panel 510, and the rear door 514. Housing feet 530A and 530B support the portable ozone generator 500 when placed on a floor or other surface. This view emphasizes the relative alignment of the upper components, the intake-side door profile, and the rear enclosure panel.

FIG. 5D illustrates an opposite side perspective 501D of the portable ozone generator 500, showing the hinged door 516 fully closed and aligned against the air intake side 506. In this embodiment, housing feet 530C and 530D are shown as alternate placements or configurations for ground-support structures. The strain relief cord connector 532 and hinge 534 are visible along the side of the housing 502, demonstrating their structural positioning and interaction with the overall enclosure design. The illustrated configuration further demonstrates how the hinged door 516 completes the front enclosure during operation and how the exterior surfaces of the portable ozone generator 500 are arranged to support airflow, cable routing, and operator access.

FIG. 5E illustrates a top-down perspective 501E of the portable ozone generator 500. In this orientation, the relative placement of the control panel 510, handle 512, hinged door 516, latches 518, rear door 514, and strain relief cord connector 532 is visible. This aerial perspective emphasizes component spacing, provides a clear representation of the device’s footprint, and illustrates how the various access panels and operational surfaces are positioned in relation to one another to form the complete enclosure layout of the portable ozone generator 500.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number, respectively. The word “or” in reference to a list of two or more items, covers all of the following interpretations of the word: any of the items in the list, all of the items in the list, and any combination of the items in the list.

Accordingly, references herein to a “fan” or “air-moving device” should be understood to encompass both bladed and bladeless airflow-generation mechanisms, including induced-flow, air-amplification, or track-based air-moving systems, as well as combinations thereof, provided that the device is capable of generating sufficient airflow to transport ambient air across the ozone generation module and distribute ozone into the treatment zone as described herein.

The phrases “in some embodiments,” “according to some embodiments,” “in the embodiments shown,” “in other embodiments,” “optionally,” and the like generally mean the particular feature, structure, or characteristic following the phrase is included in at least one implementation of the present technology and may be included in more than one implementation. In addition, such phrases do not necessarily refer to the same embodiments or different embodiments.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. Further any specific numbers noted herein are only examples, and alternative implementations may employ differing values or ranges.

The teachings of the technology provided herein can be applied to other systems, not necessarily the system described above. Further, the elements and acts of the various examples described above can be combined to provide further implementations of the technology. Some alternative implementations of the technology may include not only additional elements to those implementations noted above, but also may include fewer elements.

To reduce the number of claims, certain aspects of the technology are presented below in certain claim forms, but the applicant contemplates the various aspects of the technology in any number of claim forms. Any claims intended to be treated under 35 U.S.C. § 112(f) may begin with the words “means for” but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. A portable ozone generator comprising:
a housing defining an opening extending between an air intake side and an air discharge side;
a fan disposed within the opening of the housing and configured to draw ambient air into the air intake side and propel the ambient air toward the air discharge side;
an ozone generation module disposed within the housing and arranged around a perimeter of the opening, the ozone generation module configured to convert oxygen in the ambient air to ozone at a production rate within a range between 60 grams per hour and 384 grams per hour;
a filter positioned upstream of the ozone generation module on the air intake side;
wherein, when operated, the portable ozone generator elevates an ozone concentration within an area of up to 10,000 cubic feet to between 4 parts per million (PPM) and 70 PPM within 15 minutes to 45 minutes.

2. The portable ozone generator of claim 1, wherein the ozone generation module comprises a plurality of ozone generation units spaced equidistantly about the perimeter of the opening.

3. The portable ozone generator of claim 2, wherein the plurality of ozone generation units comprises a plurality of corona discharge ozone generation units.

4. The portable ozone generator of claim 1, wherein, when operated, the fan has a flow rate between 1800 and 4600 cubic feet per minute.

5. The portable ozone generator of claim 1, wherein, when operated, the portable ozone generator draws power from standard electrical circuitry of between 120 volts (V) and 220 V at less than 15 amps.

6. The portable ozone generator of claim 1, wherein the housing has dimensions configured for manual transport, the dimensions including:
a width of between 15 inches to 35 inches;
a height of between 15 inches to 35 inches;
a depth of between 5 inches to 15 inches; and
a weight of up to 50 pounds.

7. The portable ozone generator of claim 1, wherein the housing comprises a hinged door on the air intake side, wherein the hinged door securely attaches to a first side of the housing and permanently attaches to a second side of the housing.

8. The portable ozone generator of claim 1, further comprising a control system for maintaining the ozone concentration within the area at a preset concentration, wherein the control system comprises an ozone sensor, a run timer, and a toggle switch for selecting from a plurality of fan speed settings.

9. The portable ozone generator of claim 8, wherein the control system further comprises a wireless communication module configured to enable communication via at least one of Bluetooth, WiFi, or a local area network, wherein the wireless communication module is configured to enable coordination between a plurality of portable ozone generators deployed within a facility.

10. A method for treating an enclosed area with ozone, the method comprising:
positioning a portable ozone generator within an area, the portable ozone generator comprising a housing defining an opening extending between an air intake side and an air discharge side, a fan disposed within the opening, and an ozone generation module arranged around a perimeter of the opening;
drawing ambient air into the air intake side of the housing using the fan;
filtering the ambient air upstream of the ozone generation module;
converting oxygen in the ambient air to ozone using the ozone generation module at a production rate within a range between 60 grams per hour and 384 grams per hour;
propelling ozone-containing air from the air discharge side into the area; and
elevating an ozone concentration within the area of up to 10,000 cubic feet to between 4 parts per million (PPM) and 70 PPM within 15 minutes to 45 minutes.

11. The method of claim 10, wherein the ozone generation module comprises a plurality of ozone generation units spaced equidistantly about the perimeter of the opening.

12. The method of claim 11, wherein the plurality of ozone generation units comprises a plurality of corona discharge ozone generation units.

13. The method of claim 10, wherein, when operated, the fan has a flow rate between 1800 and 4600 cubic feet per minute.

14. The method of claim 10, wherein, when operated, the portable ozone generator draws power from standard electrical circuitry of between 120 volts (V) and 220 V at less than 15 amps.

15. The method of claim 10, wherein the housing has dimensions configured for manual transport, the dimensions including:
a width of between 15 inches to 35 inches;
a height of between 15 inches to 35 inches;
a depth of between 5 inches to 15 inches; and
a weight of up to 50 pounds.

16. The method of claim 10, wherein the portable ozone generator further comprises a control system including a wireless communication module configured to enable communication via at least one of Bluetooth, WiFi, or a local area network, wherein the wireless communication module is configured to enable coordination between a plurality of portable ozone generators deployed within a facility.

17. A portable ozone generator comprising:
a housing defining an opening extending between an air intake side and an air discharge side;
a fan disposed within the opening of the housing and configured to draw ambient air into the air intake side and propel the ambient air toward the air discharge side;
a plurality of ozone generation units disposed within the housing and arranged around a perimeter of the opening, the plurality of ozone generation units being configured to convert oxygen in the ambient air to ozone, wherein the ambient air limits a maximum ozone concentration producible by the portable ozone generator; and
wherein the portable ozone generator is configured to achieve an ozone concentration sufficient for microbial inactivation within a treatment zone.

18. The portable ozone generator of claim 17, further comprising a filter positioned upstream of the plurality of ozone generation units on the air intake side.

19. The portable ozone generator of claim 17, wherein the plurality of ozone generation units comprises a plurality of corona discharge ozone generation units.

20. The portable ozone generator of claim 17, wherein the fan is configured to move ambient air from the air intake side to the air discharge side at a rate of between 1800 and 4600 cubic feet per minute.
